(19) United States
(12) Patent Application Publication
(10) Pub. No.: US 2021/0319519 A1
(43) Pub. Date: Oct. 14, 2021

(54) CLAIM MANAGEMENT SYSTEM AND METHOD FOR PROCESSING LEGAL CLAIMS IN THE PERSONAL INJURY INDUSTRY

(71) Applicant: 15 De Novo, Inc, Los Angeles, CA (US)

(72) Inventor: Alexander Tsao, Los Angeles, CA (US)

(21) Appl. No.: 17/228,594

(22) Filed: Apr. 12, 2021

Related U.S. Application Data
(60) Provisional application No. 63/009,245, filed on Apr. 13, 2020.

(51) Int. Cl. G06Q 40/08; G16H 15/00; G06Q 50/18; G06F 40/166

(52) U.S. Cl. CPC G06Q 40/08; G06F 40/166; G06Q 50/18; G16H 15/00

(57) ABSTRACT

The present invention relates generally to a claim management system and method for processing legal claims in the personal injury industry, including medical and insurance related administrative procedures. More so, the claim management system and method provides an innovative portal for a user to digitally process a claim through guided workflows generating detailed tasks and documents tailored for that claim as well as tracking claim information and progress, facilitating open communications and document sharing between all involved parties to a claim. Through key modules within the claim management system and method, a robust framework is created to revolutionize personal injury industry standards through reductions in legal fees, decreases in claim duration, and ultimately, fostering an environment of transparency and trust between all parties at an unprecedented level.

### CROSS-REFERENCE TO RELATED APPLICATIONS

This non-provisional application claims priority under 35 U.S.C. § 119(a) on U.S. Provisional Patent Application No. 63/009,245 filed on Apr. 13, 2020, the entire contents of which are hereby incorporated by reference.

### FIELD OF THE INVENTION

The present invention relates generally to a claim management system and method for processing legal claims in the personal injury industry, including medical and insurance related administrative procedures. More so, the claim management system and method provides an innovative portal for a user to digitally process a claim through guided workflows generating detailed tasks and documents tailored for that claim as well as tracking claim information and progress, facilitating open communications and document sharing between all involved parties to a claim. Through key modules within the claim management system and method, a robust framework is created to revolutionize personal injury industry standards through reductions in legal fees, decreases in claim duration, and ultimately, fostering an environment of transparency and trust between all parties at an unprecedented level.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In recent years, the demand for professional technical consultants has increased every year. At the same time, due to the development of the Internet, an increasing number of people are frequently seeking answers or help online to get the fastest and most effective assistance when they encounter problems in their everyday life. However, due to the popularization of the Internet, the number of search engines, blogs, and forums has also significantly increased on a yearly basis. For users, it is indeed convenient to find information as the ease of access to information becomes more widely spread. However, there are still many specific and complex questions that cannot be answered directly due to peoples' inability to easily filter large amounts of information as it continues to increase. Further, people may also lack the expertise that is often needed to understand and apply said information found. Therefore, a need for professionals with extensive experience and knowledge to provide relevant answers remains essential, especially in fields of industry that deal with complex and specialized knowledge that require a particular level of expertise.

For example, when a person needs to find information after they are involved in an accident that results in a personal injury, the considerable amount of online information within the personal injury industry makes it difficult to find and filter what is applicable or relevant. In addition, the information found may also lead to misunderstanding as said person may possibly misinterpret statutory or case law or even standard assertions or testimonials read on legal websites. In turn, this usually leads to people becoming misinformed, making incorrect conclusions and then adopting wrong solutions. Furthermore, if a person is seeking free advice from an attorney online, it may not be easy to obtain the necessary information in a detailed manner that applies to their specific personal injury problem. In fact, it often becomes necessary to retain an attorney's services to obtain the corresponding information or advice which is inconvenient for said person as they still have to go to the attorney's physical office. Further, in other instances, due to the lack of effective information integration, every time a person needs to obtain additional information regarding another personal injury issue that they have experienced, they have to search again or retain an attorney once more who will provide corresponding assistance and advice. Clearly, it is quite inconvenient and frustrating for people when they are in a difficult situation where time is of the essence and they need to trust the information at hand. As such, this leads to an industry where people are forced to go through many hurdles in order to just get a basic understanding, much less gain the ability to directly handle their own personal injury matter. As such, the process for personal injury/tort claims is outdated, creates suspicion from all involved parties, prevents transparency, and has kept the marketplace from adjusting critical aspects such as pricing and client control due to lack of information.

In summary, the present invention aims at the deficiency of the above-mentioned conventional technology and proposes a claim management system and method for processing legal claims in the personal injury industry to effectively overcome the above-mentioned problems.

### SUMMARY

In order to solve the problem above, the inventor provides a method for processing legal claims in the personal injury industry, the method comprising:
accessing, by a claimant, a client portal through a software application, the claimant being involved in a personal injury incident;
recording, by the claimant, a personal injury case through a case detail module, recorded information documenting the personal injury incident;
scheduling a medical treatment calendar module to schedule medical treatment for the claimant;
documenting, by the claimant, physical and mental pain through a pain journal module;
communicating, between the claimant and a firm, through an update request module;
recording events related to the personal injury incident through an events module;
generating at least one document through a document module to generate workflow and documents between the claimant and another party such as an insurance company;
delivering, between the claimant, the insurance company, and the firm, the documents; and
awarding points to the claimant for utilizing the software application through a rewards module, the points being exchangeable for a prize;
wherein the case detail module, the medical treatment calendar module, the pain journal module, the update request module, the events module, the document module, and the rewards module are set in at least one processor.

In another aspect, the claimant accesses the client portal 24/7.

In another aspect, the document includes at least one of the following: a claimant medical record, a financial record, and an insurance claim.

In another aspect, the method further comprising: viewing the documents by the claimant through a users module, and other involved parties through shared document portal, wherein the users module is also set in the at least one processor.

In addition, the inventor also provides a claim management system for processing legal claims in the personal injury industry, the system comprising:

At least one processor configured to:
accessing, by a claimant, a client portal through a software application, the claimant being involved in a personal injury incident;
recording, by the claimant, a personal injury case, recorded information documenting the personal injury incident;
scheduling medical treatment for the claimant;
documenting, by the claimant, physical and mental pain;
communicating between the claimant and a firm;
recording events related to the personal injury incident;
generating at least one document to generate workflow and documents between the claimant and another party such as an insurance company;
delivering, between the claimant, the insurance company, and the firm, the documents; and
awarding points to the claimant for utilizing the software application, the points being exchangeable for a prize.

In another aspect, the claimant accesses the client portal 24/7.

In another aspect, the document includes at least one of the following: a claimant medical record, a financial record, and an insurance claim.

In another aspect, the at least one processor further configured to viewing the documents by the claimant and other involved parties through shared document portal.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1A illustrates a screenshot of a communication device, showing an exemplary system for processing legal claims in the personal injury industry, including medical and insurance related administrative procedures, in accordance with an embodiment of the present invention;

FIG. 1B illustrate screenshots of an exemplary client portal, showing a client portal module, in accordance with an embodiment of the present invention;

FIGS. 2A-2D illustrate screenshots of an exemplary client portal, showing a case detail module, in accordance with an embodiment of the present invention;

FIG. 3 illustrates a screenshot of an exemplary client portal, showing a medical treatment calendar module, in accordance with an embodiment of the present invention;

FIG. 4A illustrates a screenshot of an exemplary client portal, showing a pain journal module, in accordance with an embodiment of the present invention;

FIG. 4B illustrates a screenshot of an exemplary client portal, showing an add entry module, in accordance with an embodiment of the present invention;

FIG. 5A illustrates a screenshot of an exemplary client portal, showing an update request module, in accordance with an embodiment of the present invention;

FIG. 5B illustrates a screenshot of an exemplary client portal, showing an update request details module, in accordance with an embodiment of the present invention;

FIG. 6 illustrates a screenshot of an exemplary client portal, showing an events module, in accordance with an embodiment of the present invention;

FIG. 7A illustrates a screenshot of an exemplary client portal, showing a document module, in accordance with an embodiment of the present invention;

FIG. 7B illustrates a screenshot of an exemplary client portal, showing an add new document module, in accordance with an embodiment of the present invention;

FIG. 8 illustrates a screenshot of an exemplary client portal, showing a notification module, in accordance with an embodiment of the present invention;

FIG. 9A illustrates a screenshot of an exemplary client portal, showing a rewards module, in accordance with an embodiment of the present invention;

FIG. 9B illustrates a screenshot of an exemplary client portal, showing an award of 100 points in the rewards module, in accordance with an embodiment of the present invention;

FIG. 10A illustrates a screenshot of an exemplary client portal, showing a settings module, in accordance with an embodiment of the present invention;

FIG. 10B illustrates a screenshot of an exemplary client portal, showing a chat module, in accordance with an embodiment of the present invention;

FIG. 11A illustrates a screenshot of an exemplary law firm portal, showing a dashboard module, in accordance with an embodiment of the present invention;

FIG. 11B illustrates a screenshot of an exemplary law firm portal, showing a master settings module for the listing of workflow templates, in accordance with an embodiment of the present invention;

FIG. 11C illustrates a screenshot of an exemplary law firm portal, showing a master settings module for a workflow template, in accordance with an embodiment of the present invention;

FIG. 11D illustrates a screenshot of an exemplary law firm portal, showing a master settings module for the task templates within a workflow template, in accordance with an embodiment of the present invention;

FIG. 11E illustrates a screenshot of an exemplary law firm portal, showing a master settings module for settings of a task template within a workflow template, in accordance with an embodiment of the present invention;

FIG. 11F illustrates a screenshot of an exemplary law firm portal, showing a matter module for a listing of all matters, in accordance with an embodiment of the present invention;

FIG. 11G illustrates a screenshot of an exemplary law firm portal, showing a matter module for a listing of all tasks, in accordance with an embodiment of the present invention;

FIG. 11H illustrates a screenshot of an exemplary law firm portal, showing a case details module for a matter, in accordance with an embodiment of the present invention;

FIG. 11I illustrates a screenshot of an exemplary law firm portal, showing a workflow module for the listing of generated workflows, in accordance with an embodiment of the present invention;

FIG. 11J illustrates a screenshot of an exemplary law firm portal, showing a workflow module for generating workflow, in accordance with an embodiment of the present invention;

FIG. 11K illustrates a screenshot of an exemplary law firm portal, showing a tasks module for tasks generated in a matter, in accordance with an embodiment of the present invention;

FIG. 11L illustrates a screenshot of an exemplary law firm portal, showing a shared documents module for documents to be shared with other parties, in accordance with an embodiment of the present invention;

FIG. 11M illustrates a screenshot of an exemplary law firm portal, showing an users module for a listing of all clients, in accordance with an embodiment of the present invention;

FIG. 12 illustrates a flowchart of an exemplary method for processing legal claims in the personal injury industry, including medical and insurance related administrative procedures, in accordance with an embodiment of the present invention;

FIGS. 13A-13F illustrate diagrams and flowcharts for the client portal in a business/enterprise version, in accordance with an embodiment of the present invention;

FIGS. 14A-14C illustrate diagrams and flowcharts for the client portal in an individual version, in accordance with an embodiment of the present invention;

FIG. 15 illustrates a diagram of a Dynamic Client Database Tool that allows effective case processing and management while simultaneously allowing designated third parties the ability to view case progress and information, in accordance with an embodiment of the present invention;

FIG. 16 illustrates a flowchart for the business and enterprise versions of the law firm portal and client portal, in accordance with an embodiment of the present invention; and

FIG. 17 illustrates a flowchart for the individual version of the client portal, in accordance with an embodiment of the present invention.

Like reference numerals refer to like parts throughout the various views of the drawings.

### DETAILED DESCRIPTION OF THE INVENTION

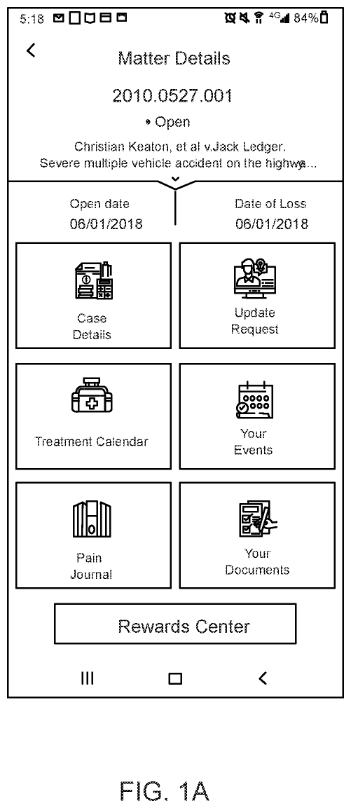

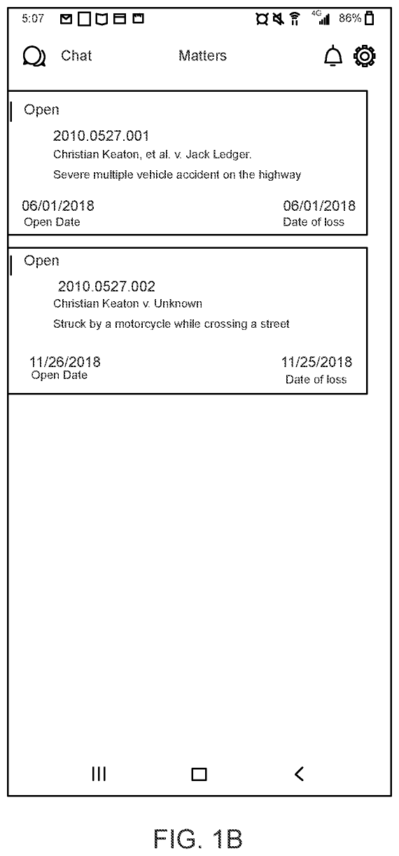

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 105 U.S.C. § 112.

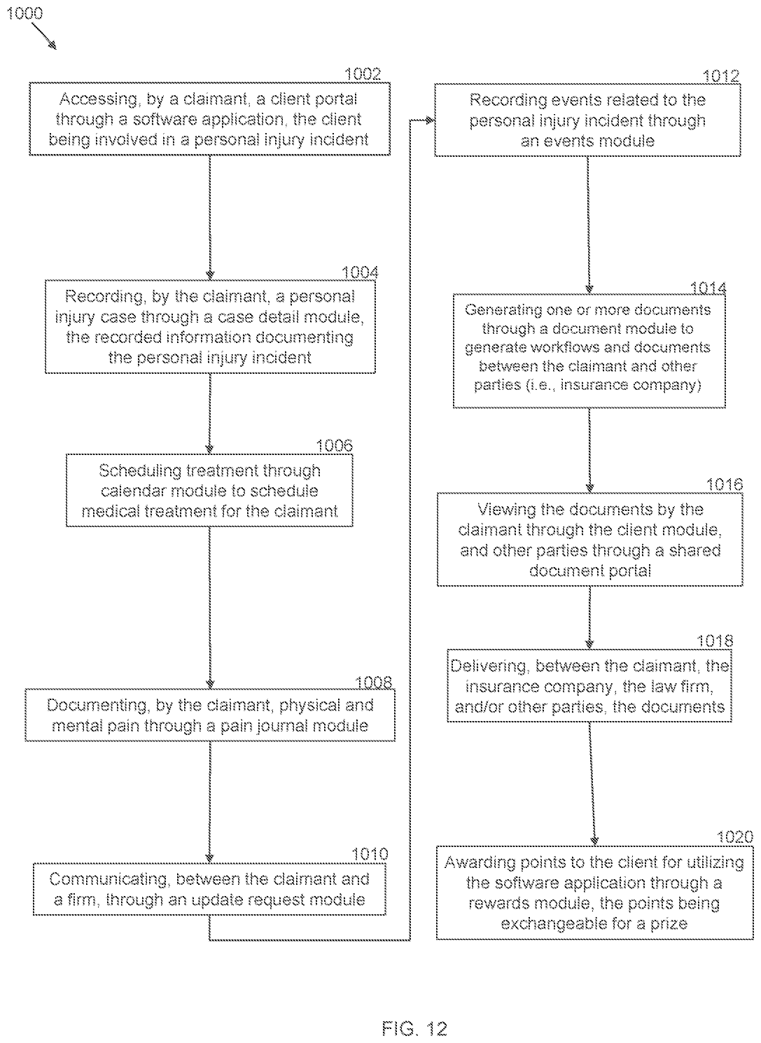

In one embodiment of the present invention presented in FIGS. 1-17, a system 100 and method 1000 for processing legal claims in the personal injury industry, including medical and insurance related administrative procedures, provides an innovative portal for a user to digitally process a claim through guided workflows generating detailed tasks and documents tailored for that claim as well as tracking claim information and progress, facilitating open communications and document sharing between all involved parties to a claim. The system 100 and method 1000 also serves to create a robust framework in order to revolutionize personal injury industry standards through reductions in legal fees, decreases in claim duration, and ultimately, fostering an environment of transparency and trust between all parties. The users access the portal through a software application that displays multiple key modules

In some embodiments, the modules may include: a case details module 110 to record and document specific information 24/7; a medical treatment calendar module 120 to schedule medical treatment for the claimant; a pain journal module 130 to document physical and mental pain of the claimant; an update request module 140 for communicating with administrators; an events module 150 to record events; and a document module 160 to facilitate generation of workflow and documents, which decrease operational load and allows for more attorney-client compensation structures. A rewards module 180 awards points to the claimant to encourage usage of the software application.

As FIG. 1A shows, the system 100 is unique in allowing a claimant, an insurance company, and a firm to process medical-related and insurance-related administrative procedures. In usage, the claimant can check the status of insurance claims anytime (24/7), record medical status, record insurance status, communicate with a case specialist to ask questions, upload and download documents for sharing with the insurance company, and receive claim documents and communications from the firm and the insurance company. Additionally, through the client portal, the claimant can write record medical issues in a pain journal module 130, which is utilized to document an injury experience. Also, included in the system 100 and method 1000 is shared document portal, usable by the insurance company, for secured transfer of documents and files and easy access to relevant claim files shared between all parties through their respective portals.

One objective of the present invention is to provide customized client management system 100 that digitizes the personal injury process and creates value on multiple sides of a claim.

Another objective is to allow the claimant access to personal injury incident 24/7.

Yet another objective is to allow the claimant, insurance company, and the firm to communicate seamlessly.

An exemplary objective is to allow the claimant, insurance company, and the firm to exchange and view personal injury incident documents.

Additional objectives are to provide an easy to use client portal, law firm portal, shared portal or processing medical and insurance related processes in a personal injury claim.

FIG. 1A is a flowchart of an exemplary method 1000 for processing legal claims in the personal injury industry, including medical and insurance related administrative procedures. The method 1000 may include an initial Step 1002 of accessing, by a client, a client portal through a software application, the client being involved in a personal injury incident. The personal injury incident may include an automobile accident, a medical injury/disease, and/or a general insurance claim. The claimant operates the method 1000 through the client portal, which is accessible through a software application downloaded on the claimant's communication device, i.e., smart phone, laptop.

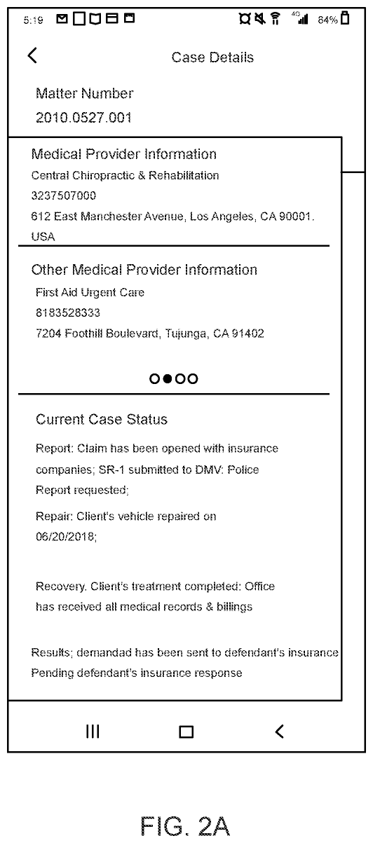

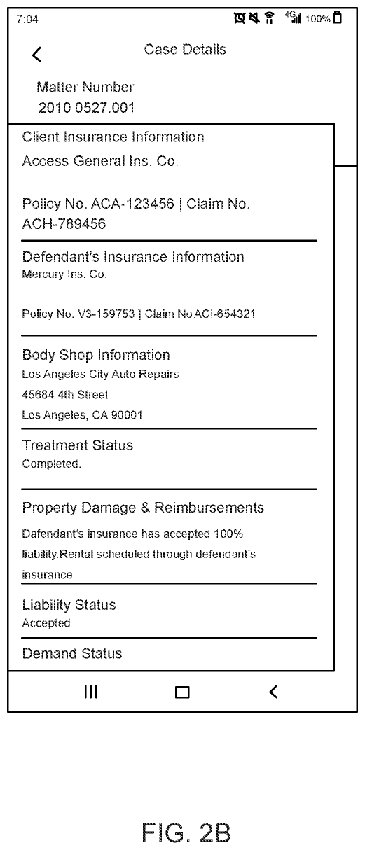

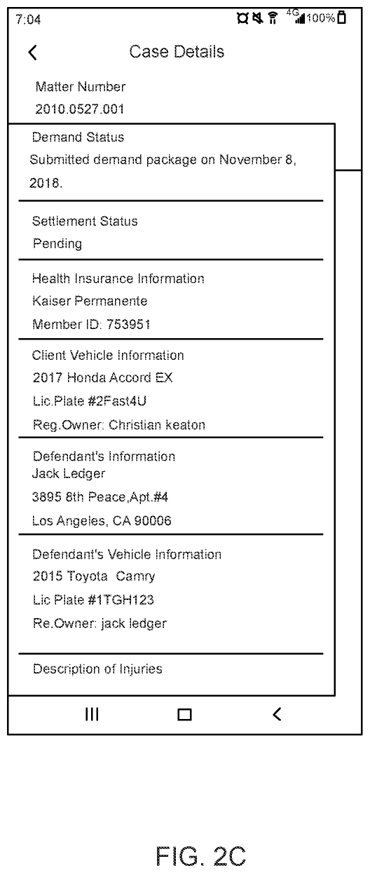

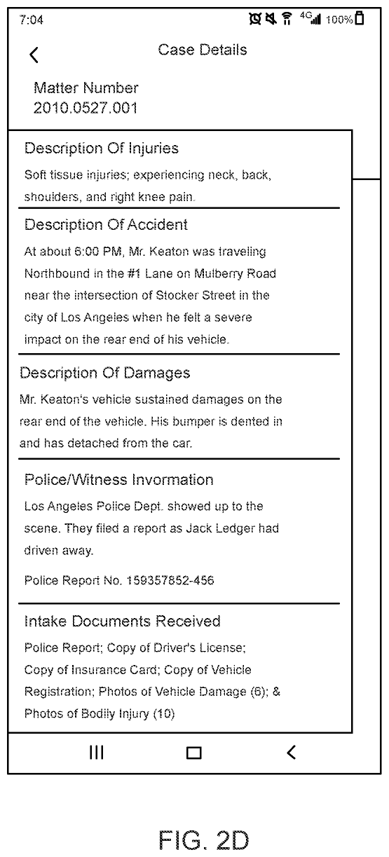

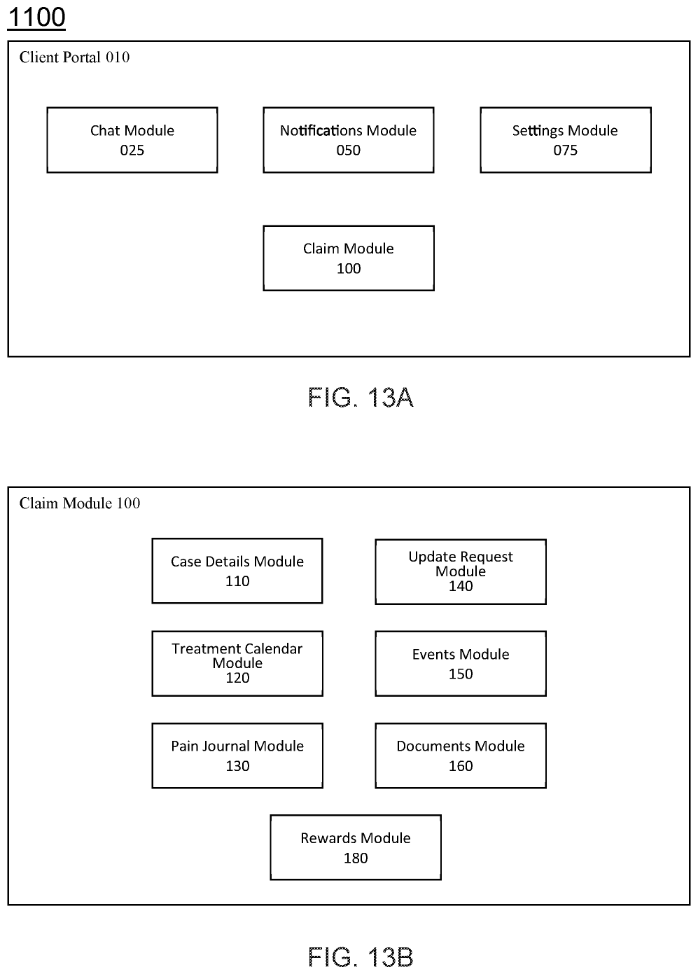

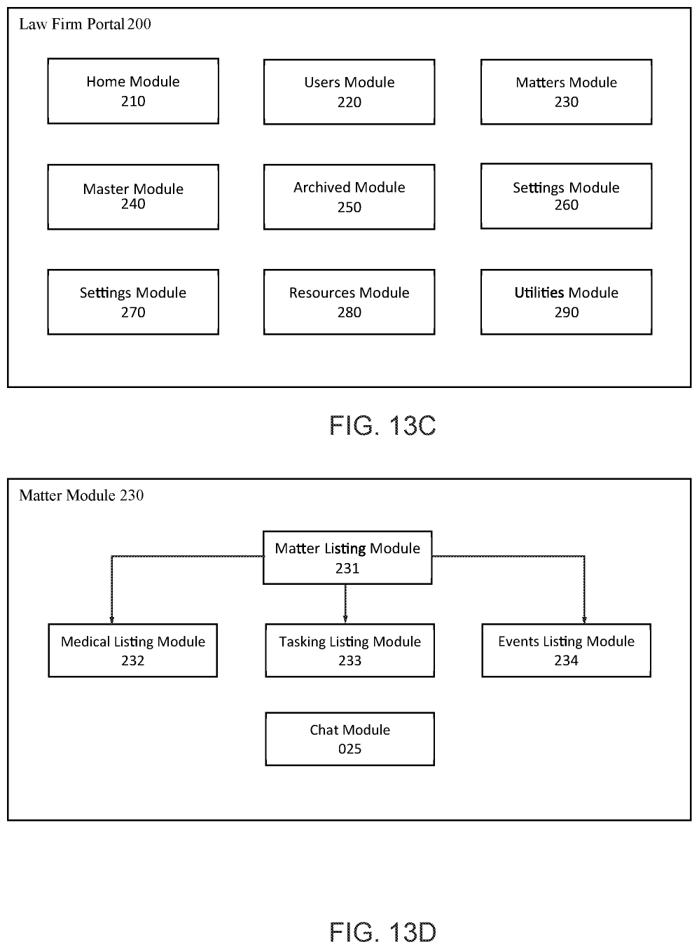

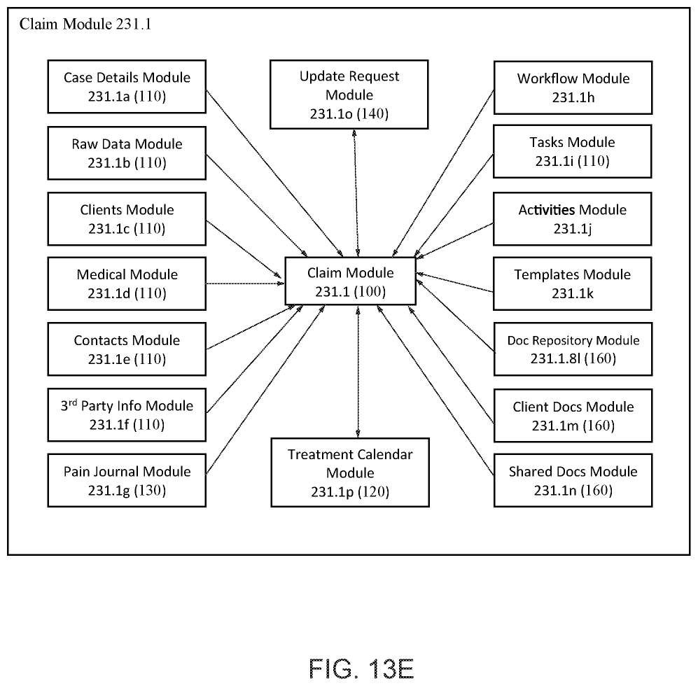

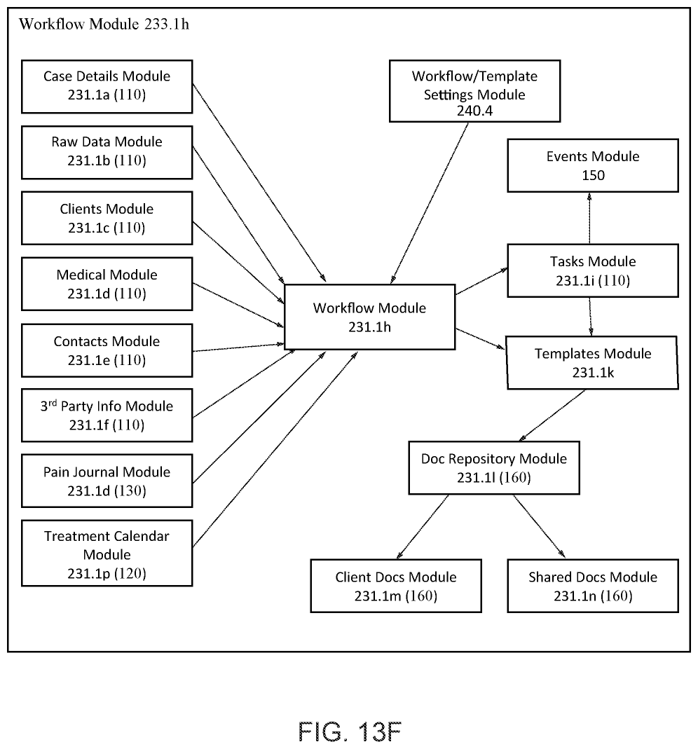

Turning now to FIGS. 2A-2D, the method 1000 also includes a Step 1004 of recording, by the claimant, a personal injury case through a case detail module 110, the recorded information documenting the personal injury incident. The case detail module 110 allows the claimant to input information, and thereby record personal injury incidents for future reference by the claimant, the insurance company, and the firm. This is an all-encompassing module that provides a snap shot of the claimant's status. A case detail module 110 also displays information that was added/changed from a claim module 231.1 as shown in FIG. 13E.

FIG. 2A shows that the case detail module 110 includes a Medical Provider Information Section 402 that gives the name and address of the medical provider, i.e., doctor. Another section is Other Medical Provider Information Section 404, which gives the name and address for secondary medical professionals. Yet another section accessible by the claimant is a Current Case Status Section 406 that details the status of the case. This can include when the case has opened, the type of personal injury case (accident), and recovery status of the claimant.

FIG. 2B shows the case detail module 110 scrolled down to reference a Client Insurance Information Section 408, which shows the name of the insurance company and the policy number for the claimant. A Defendant's Insurance Information Section 410 shows the name of the vehicle insurance company and the policy number for the defendant, or opposing Client, if there is one. This can be useful for the vehicle insurance companies to contact each other. Yet another section of the case detail module 110 is a Body Shop Information Section 412 that shows the name and contact information for a body shop. This is useful when the medical-related incident is a vehicle accident that results in damage to the vehicle. Another section is Treatment Status Section 414 that contains information on treatment to the claimant. A Property Damage and Reimbursement Section 416 details the actions of the insurance company, and any reimbursements the claimant receives. A Liability Status Section 418 shows whether the liability is accepted.

Turning now to FIG. 2C, the case detail module 110 scrolls down to a Demand Status Section 420 that indicates the status of the demand package. This can include if a demand package is being drafted, finalized, or already sent to the insurance company. A Settlement Status Section 422 that indicates if any settlement has been reached. A Health Insurance Information Section 424 shows the name of the health insurance company and the policy number for the defendant. This can be useful for the different insurance companies to contact each other. A Client Vehicle Information Section 426 shows the registration and license plate number of the vehicle driven by the claimant. A Defendant Information Section 428 shows the contact information, i.e., address, of the defendant. Continuing with the case detail module 110, a Defendant Vehicle Information Section 430 shows the registration and license plate number of the vehicle driven by the defendant. A Description of Injuries Section 432, 432 shows the detailed information about the types and locations of physical injuries to the claimant. This can include a medical description type of incident.

Looking now at FIG. 2D, a Description of Accident Section 434 shows the detailed information about the types and locations of an accident by the claimant. Continuing with the case detail module 110, a Description of Damages Section 436 includes recorded information of any property damages to the vehicle of the claimant. A Police/Witness Information Section 438 shows any comments and reports from the police or eye witnesses. This can be useful for chronicling vehicle accidents involving the claimant. Yet another section in the case detail module 110 is an Intake Documents Received Section 440 that lists the incident related documents recorded and available. These documents and files are accessible by the claimant and law firm and if given permission, by others parties such as insurance company, medical providers.

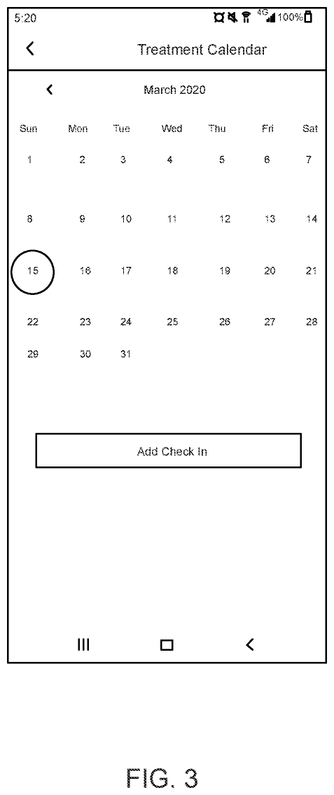

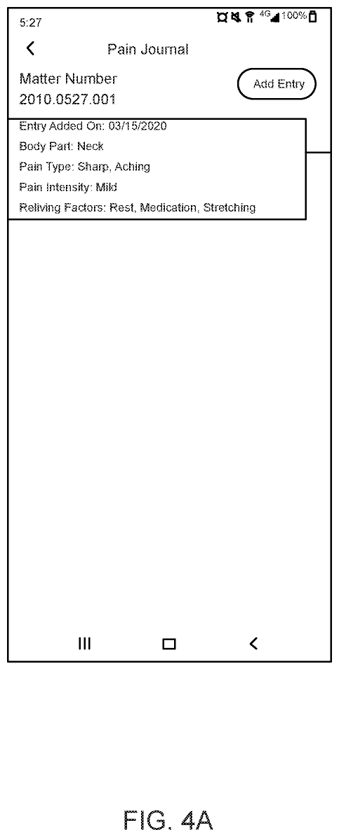

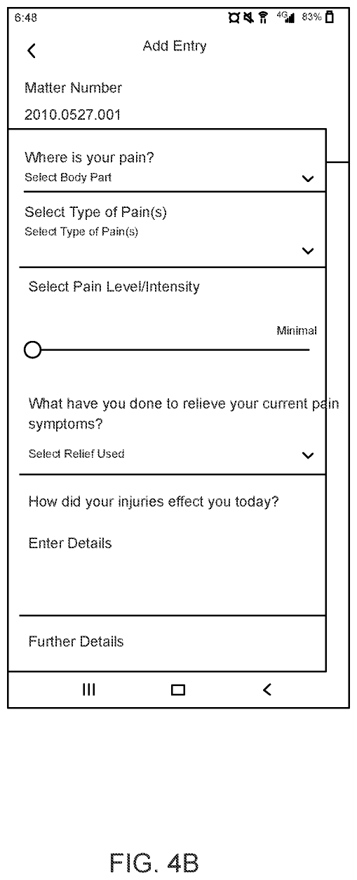

Turning now to FIG. 3, a Step 1006 provides for scheduling a medical treatment through the medical treatment calendar module 120 to schedule medical treatment for the claimant which is also accessible in module 231.1p in FIG. 13E. The claimant or medical professional can schedule times and dates for medical procedures. A notification can help remind the claimant when the scheduled day approaches. In some embodiments shown in FIG. 4A, a Step 1008 comprises documenting, by the claimant, physical and mental pain through a pain journal module 130 which is also accessible in module 231.1g in FIG. 13E. This can include a body type (head); a pain type (numbing); a pain intensity (moderate); and a relieving factor (medication). Other mental injury and pain notes may also be recorded by the claimant as they occur. As shown in FIG. 4B, is a screenshot of an exemplary client portal, showing an add entry module 131 where the Client can enter information and create the entries for pain journal module 130. For example, a drop box asking “Where is your pain?”, with the answers including various parts of the anatomy. The settings for customizing the options available in the add entry module 131 may be modified by the master module 240 within the law firm portal module 200.

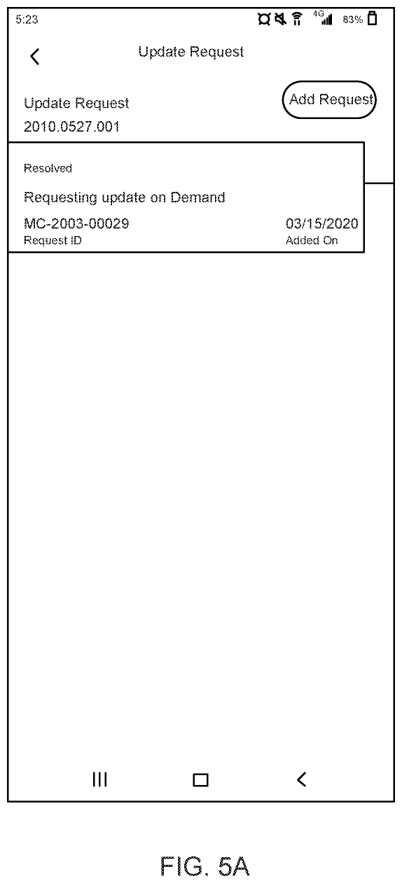

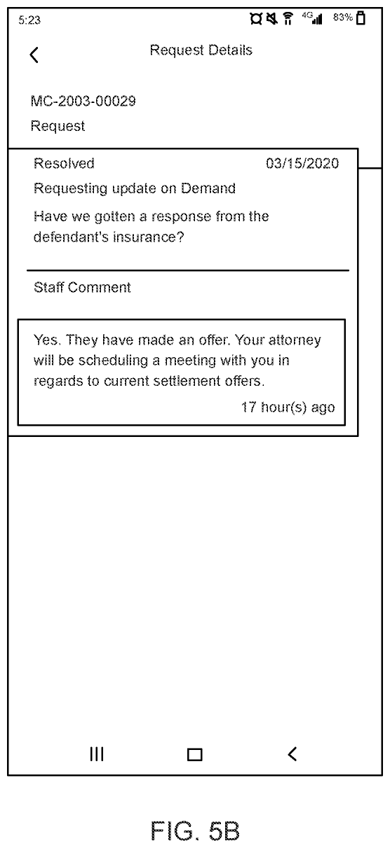

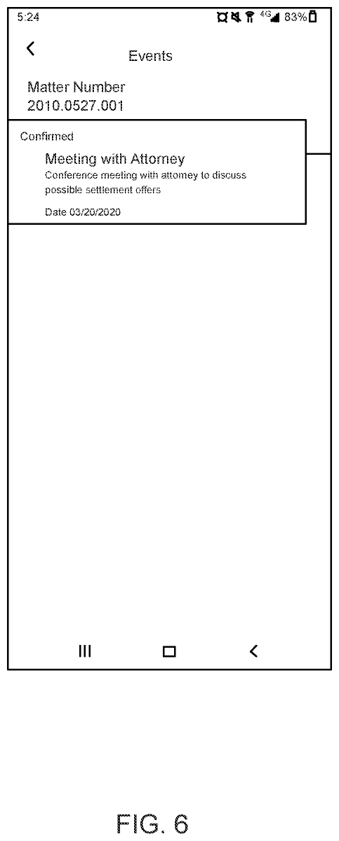

Looking now to FIG. 5A, a Step 1010 includes communicating through an update request module 140, which allows a client to request updates which are accessed by a law firm through module 231.10 shown in FIG. 13E. Another unique module is shown in FIG. 5B. This is a screenshot of an exemplary client portal, showing a request details module 141. The request details module 141 is to communicate with customer service, and archive the communication. For example, the Client asks, “Have we gotten a response from the defendant's insurance?”. The response indicates that an offer was made for the accident from the insurance. Continuing with the method 1000, a Step 1012 includes recording events related to the personal injury incident through an events module 150 to allow a client to better manage their deadlines and schedules as shown in FIG. 6. This can include any events outside of medical treatment, such as meeting with an attorney to discussion settlement offers, the date of a vehicle inspection or deadline date to submit further documentation to an insurance company.

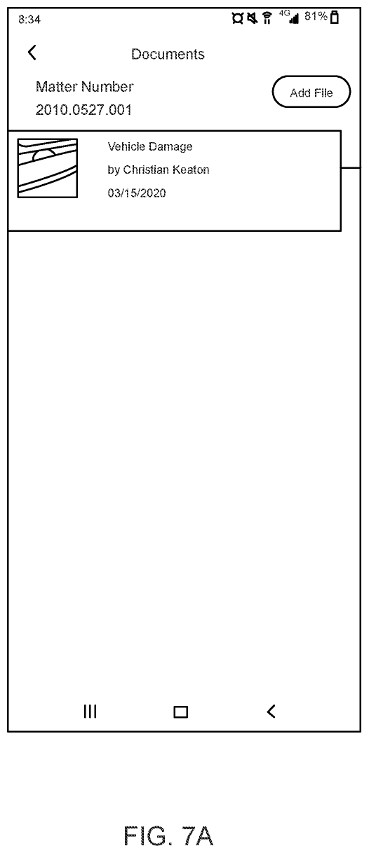

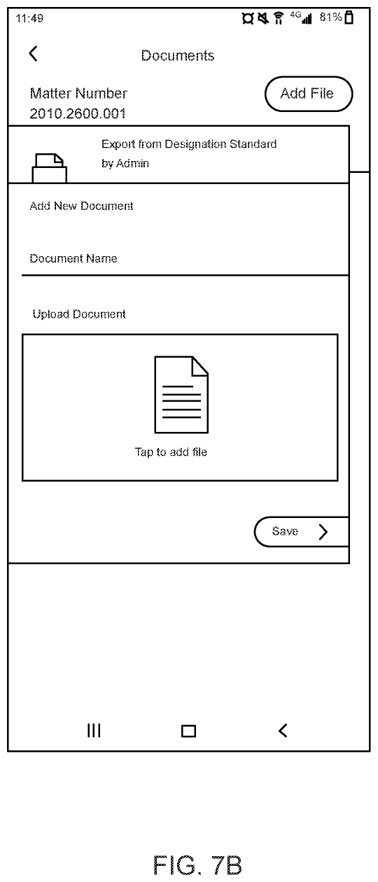

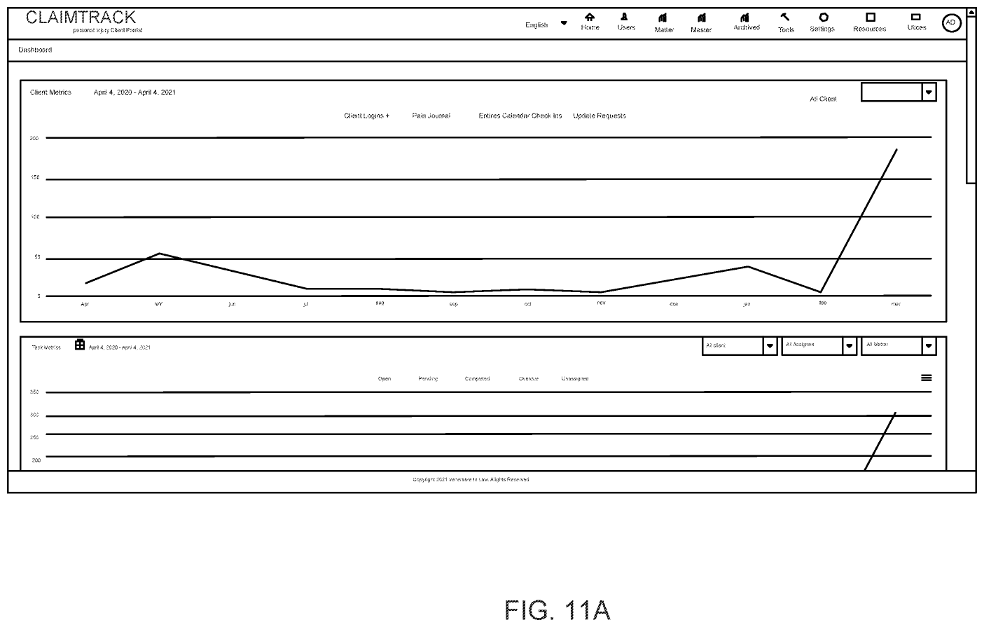

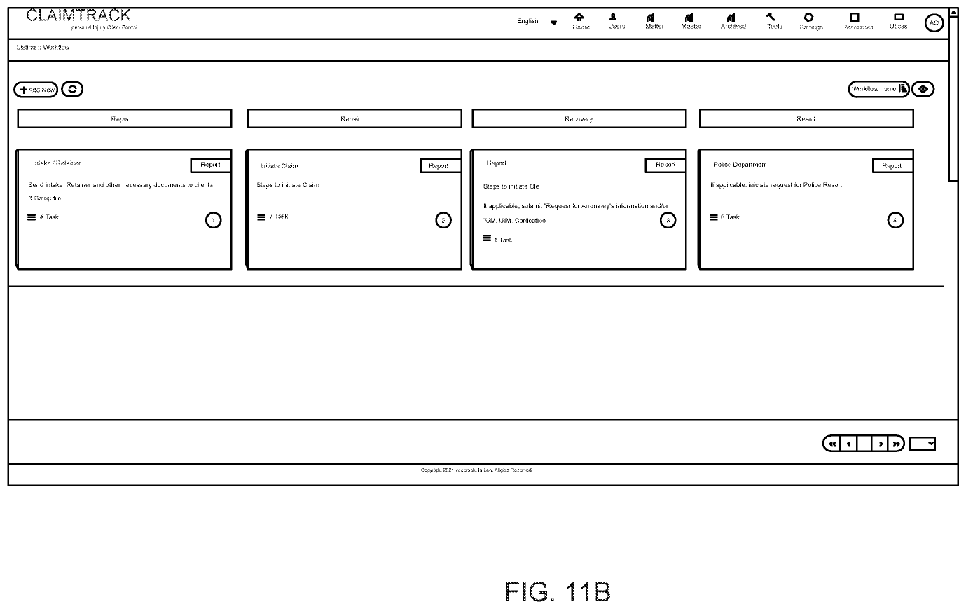

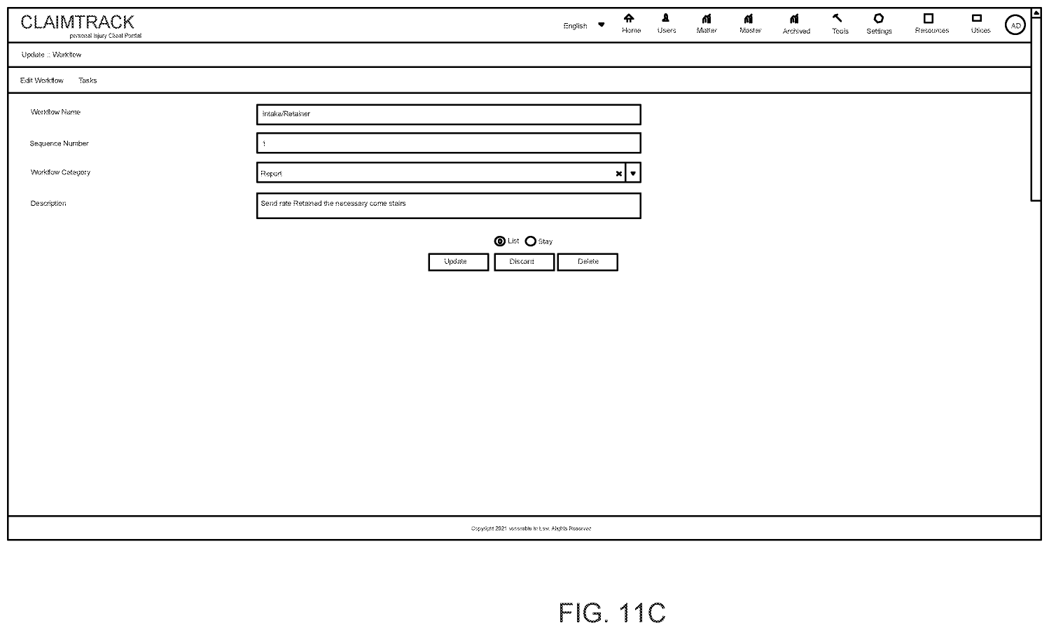

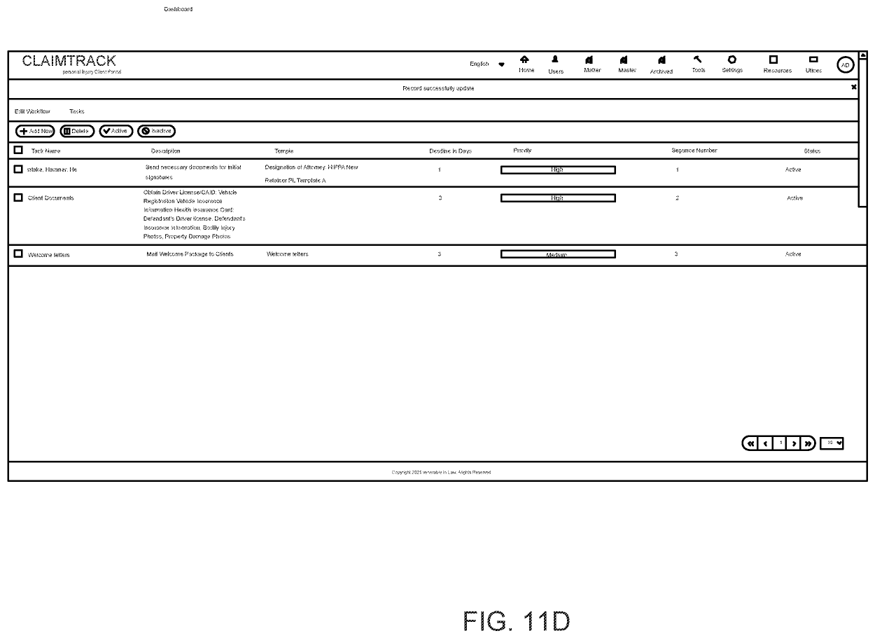

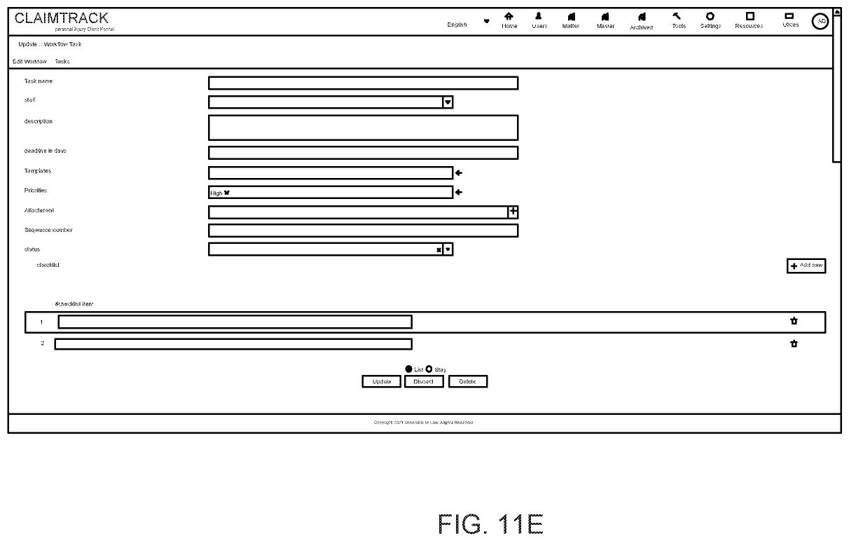

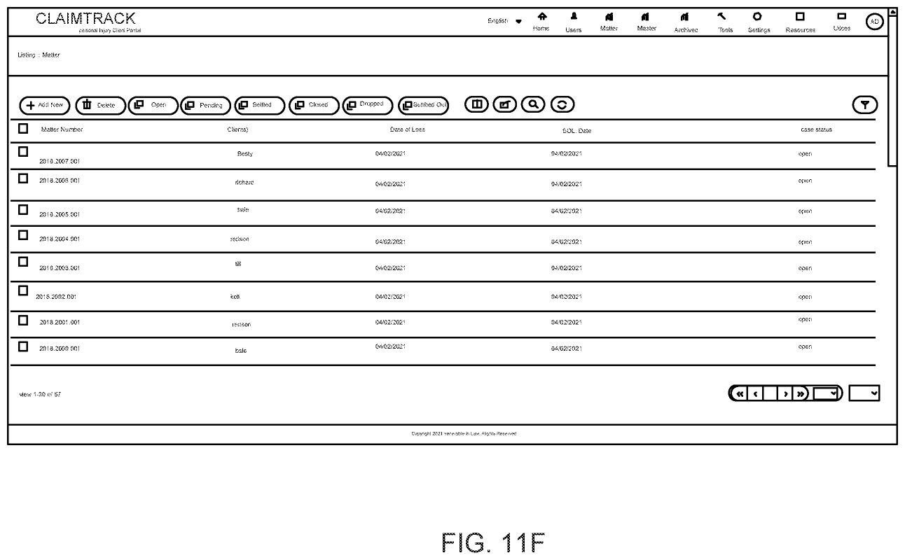

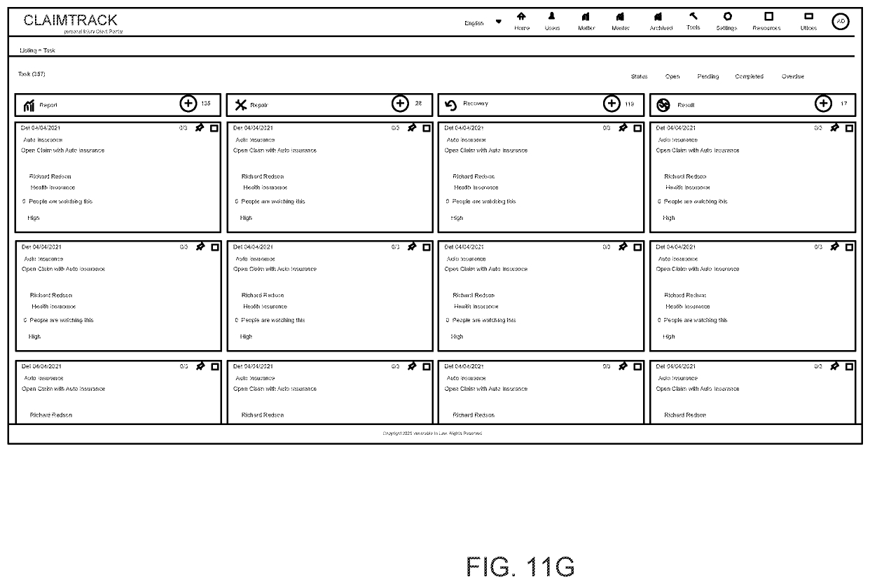

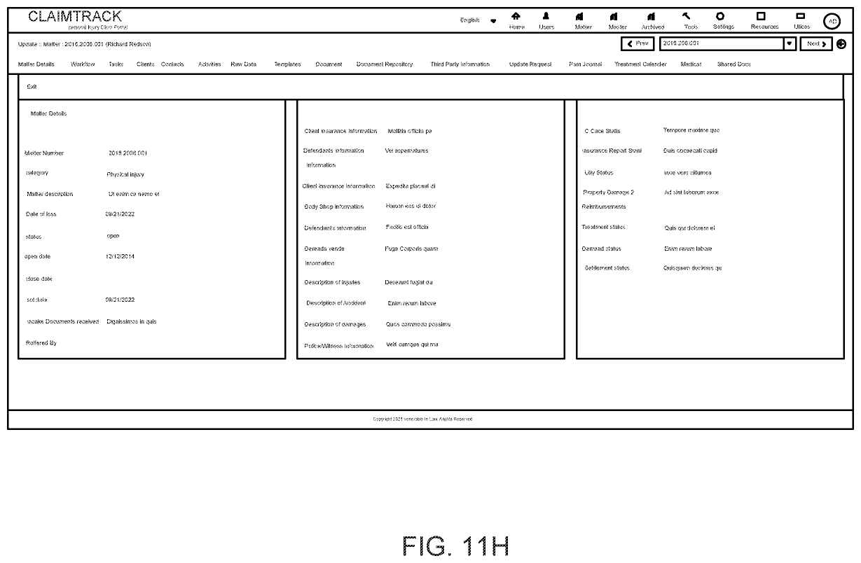

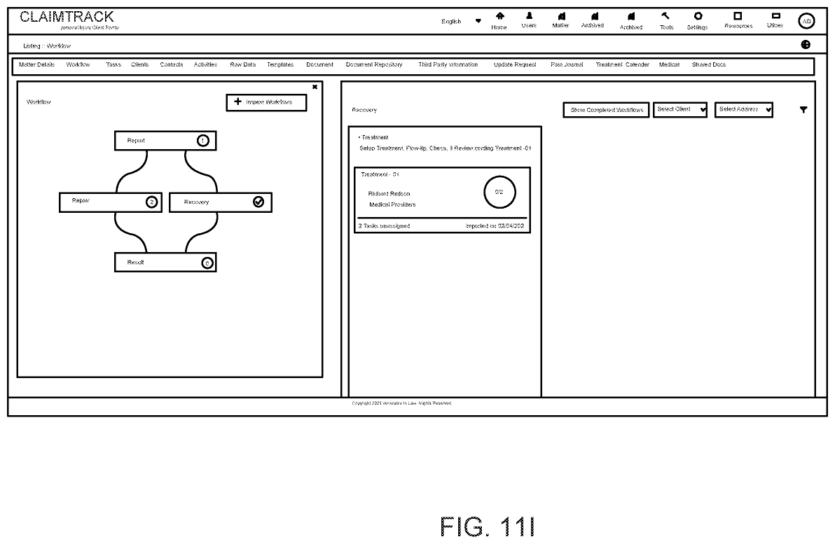

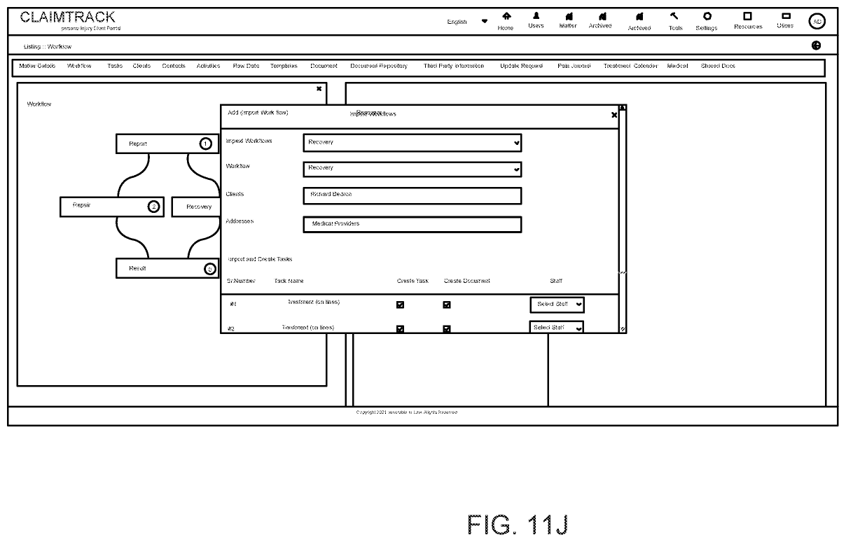

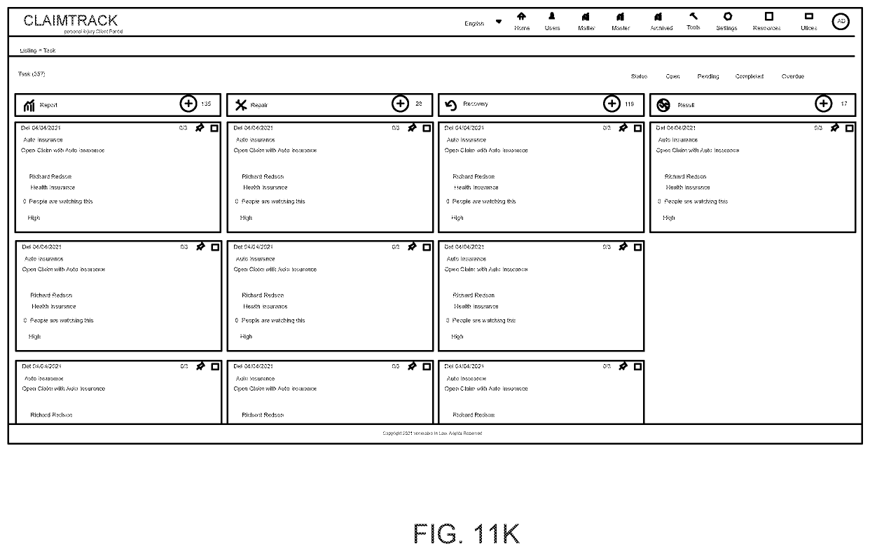

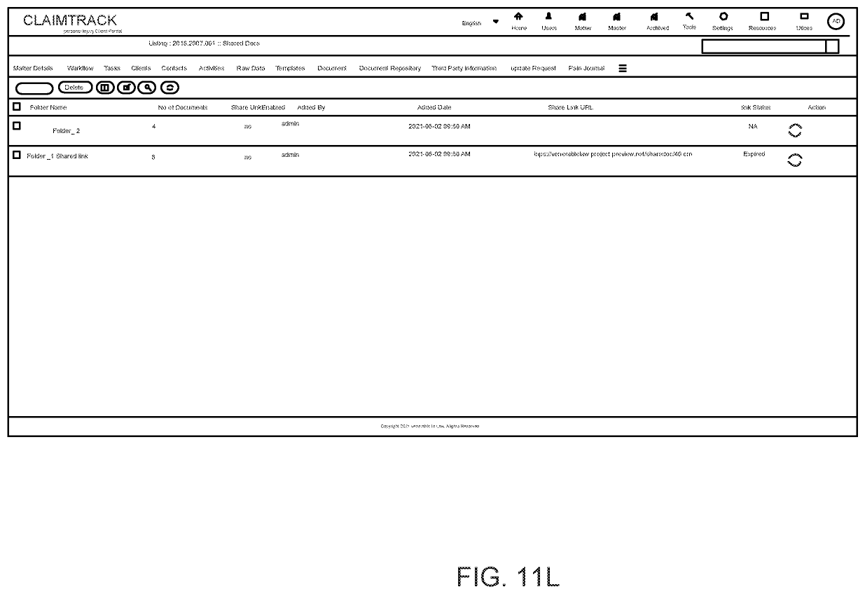

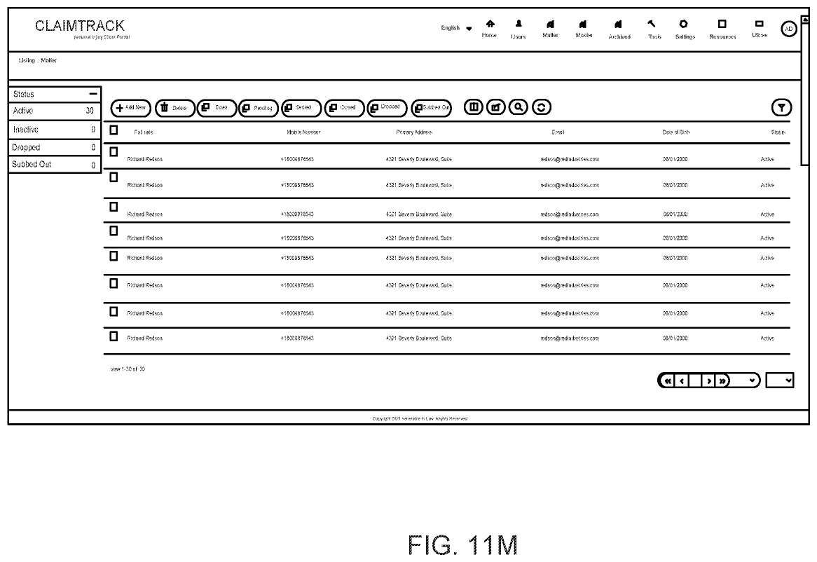

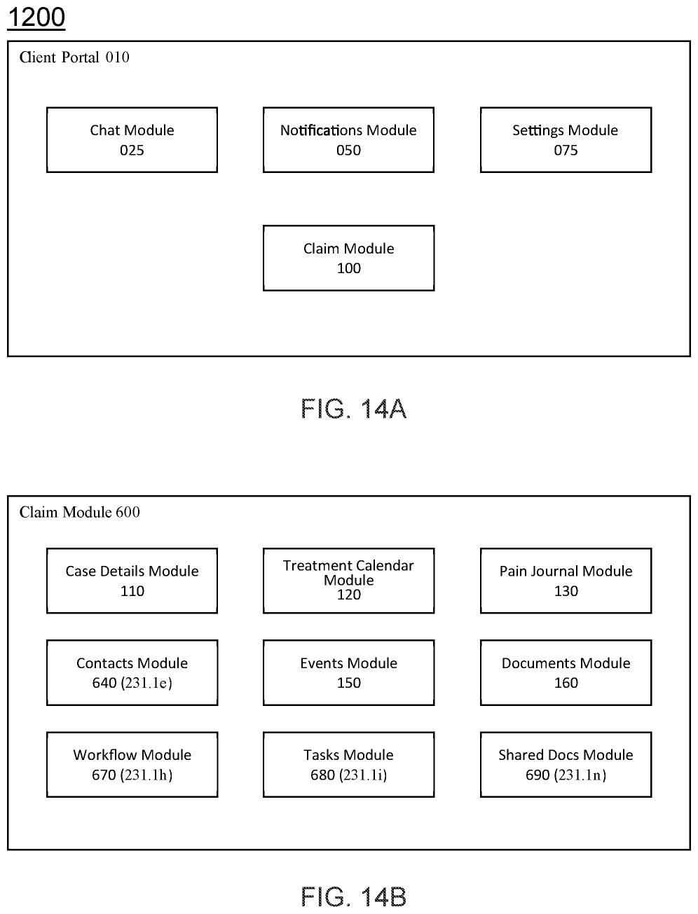

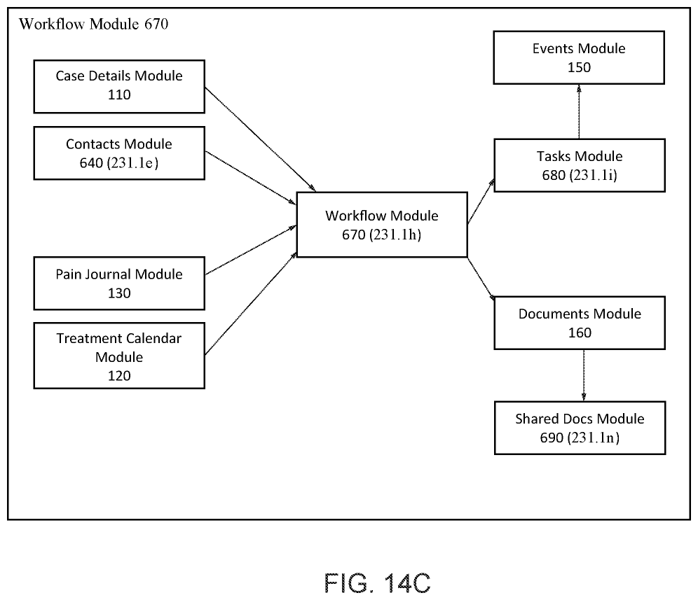

Turning now to FIG. 7A, 11I, 11J, 11K, 11L, 13E, and 14C, a Step 1014 (as illustrated in FIG. 13E and 14c) comprises generating one or more documents and/or one or more tasks through a workflow module 231.1h or 670 (shown in FIG. 111) by importing workflow and task templates from module 240.4 using data from modules 110, 120, and 130 (shown in FIG. 11J). After importing, the system will generate the tasks (shown in FIG. 11K) which may add deadline events to events module 150 as well as draft version of documents which can be exported to document module 160 or shared module 231.1n (shown in FIG. 11L). This process facilitates generation of workflow and documents between the claimant and another party (i.e., an insurance company, medical provider, or law firm). The document module 160 also allows for downloading and viewing of documents by the user, while the shared module 231.1n allows for downloading and viewing of documents by other parties such as an insurance company, medical provider and/or law firm.

Turning now to FIG. 7B, a add new document module 161 allows for uploading of documents by the claimant which can then be exported to shared document module 231.1n. From the shared document module 231.1n, all parties can view the documents, unless there is an administrative restriction. In some embodiments, the documents may include, without limitation, a claimant medical record, a financial record, and an insurance claim. For further documenting an accident, the Client can add documents such as photographs of property damage or possible ailing body parts through the add new document module 161. This allows the user to have visual proof that an incident, i.e., auto crash has occurred.

The method 1000 may further comprise a Step 1016 of downloading and viewing of documents by the claimant through the document module 160, and downloading and viewing of documents by other parties such as an insurance company, medical provider and/or law firm through the shared module 231.1n which can be accessed through a shared document portal. A Step 1018 includes delivering, between the claimant, the insurance company, the law firm, and/or other parties, the documents. This can be through a server or a cloud that receives and transmits the documents and files.

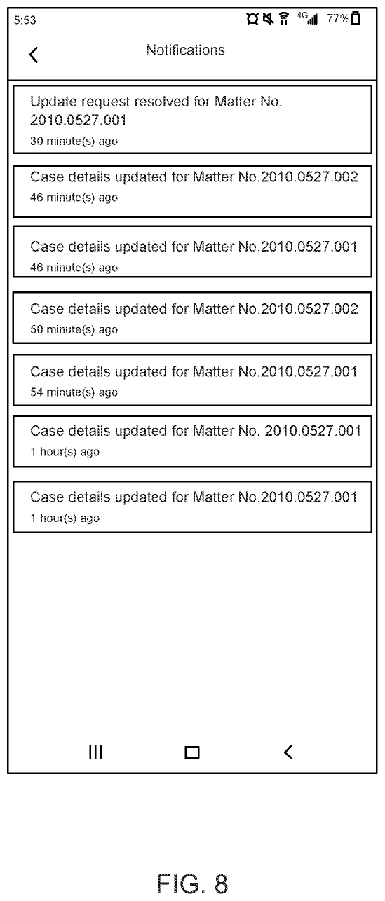

Turning now to FIG. 8 shows, the claimant can receive notifications through a notification module 050. This can be, for example, that the pain injury journal module is missing. The notifications help keep the claimant on track with their personal injury claim.

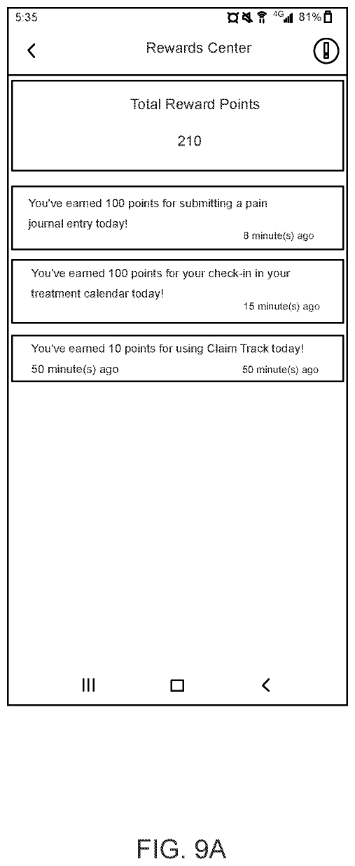

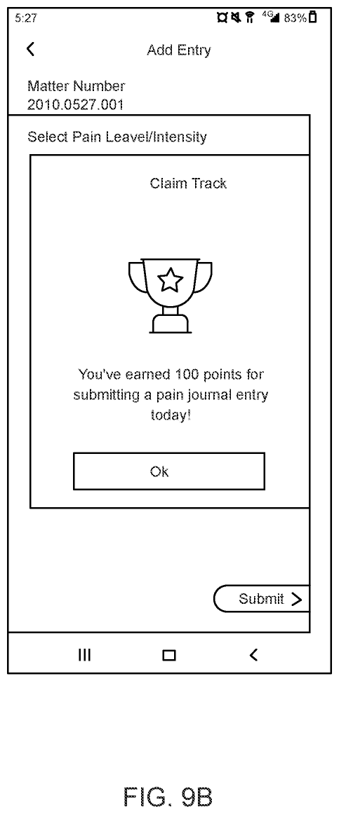

As FIG. 8A shows, a final Step 1020 includes awarding points to the claimant for utilizing the software application through a rewards module 180, the points being exchangeable for a prize. The points encourage the client to access the software application. For example, the illustration of the rewards module 180 provides 30 points for using the software application four times in one week. And awarding 10 points for using the software application in a specific day. FIG. 9B illustrates a screenshot of an exemplary client portal 181, showing an award of 100 points in the rewards module for submitting a pain journal entry, for example. If the claimant needs to change settings, this is possible in a settings module 075, shown in FIG. 9A. The settings module 075 includes: an edit profile section, a change password section, and a contact section.

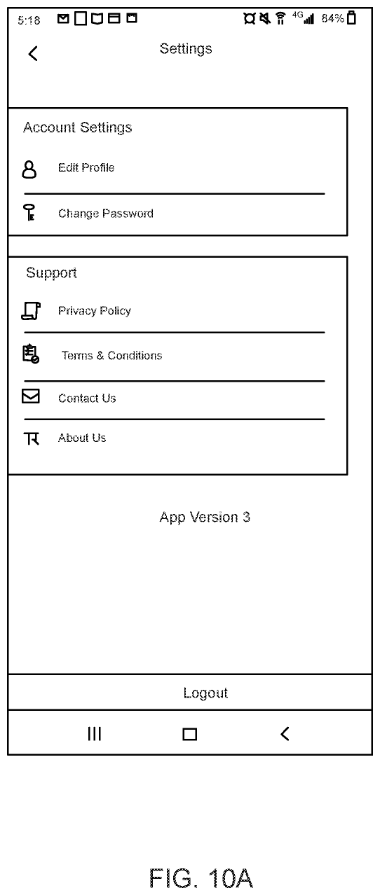

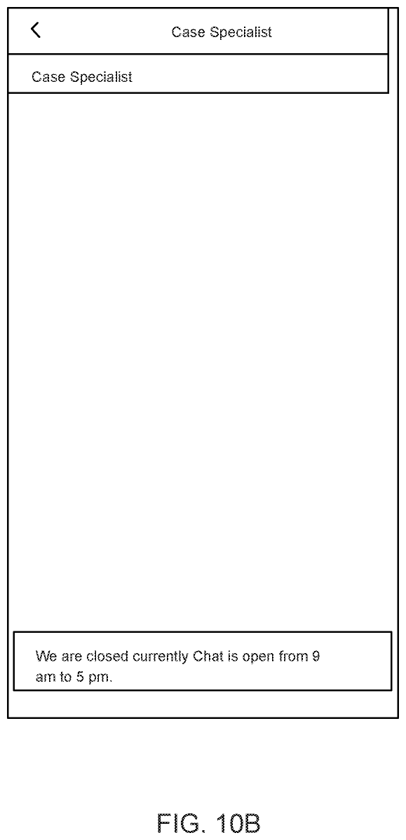

Other modules in the system are unique in providing flexibility and great functionality for the Client. As shown in FIG. 10B, a Step 1010 includes instant communications between the claimant and a case specialist during office hours, through a chat module 025.

In essence, the modules are part of a unique system 100 and method 1000 provide a customized client management system 100 that digitizes the personal injury process and creates value on multiple sides of a claim. Through workflow guides and automated fields/document generation, the firm operations are made efficient with technology solutions. Through the portals, a 24-hour bridge of communication between the firm and the claimant is laid and claims are made transparent inviting more claimant participation through pain journals and medical tracking. Consequently, trust with necessary parties, such as an insurance company, is also increased with shared document portals where documents are made available in real time, preventing unnecessary delays in processing and making communication more open.

FIGS. 13A-13F illustrate diagrams and flowcharts for the client portal in a business/enterprise version 1100. The following client portal is an outline for operation of the business/enterprise version.
Client Portal 010—Once logged in, a client can choose a specific claim. They have access to a live chat, notifications, and settings & support information.
   Claim Module (100)
      Lists all of a client's claims
   Chat Module (025)
      Allows a client to chat directly with a case specialist during office hours set by the law firm portal.
   Notifications Module (050)
      Shows a client a list of notifications of certain actions within their cases.
      Notifications also list specific automated reminders (i.e., if a client has not check-in for treatment or made a pain journal entry for a specific period, the system will send them a reminder to do so).
   Settings Module (025)
      Allows a client to edit their profile or change password as well as access to specific support information.
Claim Module 100—When a specific claim is chosen by a client, they will have access to the following modules for that claim:
   Case Details Module (110)
      Module allows a client to see their current case status as well as static data entered by the law firm from the law firm portal
   Medical Treatment Calendar Module (120)
      Client is only able to check-in when they go to treatment which is viewable by the client and the law firm.
      Client and/or the law firm will be able to set a specific schedule which the client will have to adhere to.
      Client and/or the law firm will be able to add medical appointments with necessary information such as location and notes.
   Pain Journal Module (130)
      Module lists all pain journals entry made by client and allows the client to add further entries as needed.
      A client may choose to view a specific entry for further information they added.
   Update Request Module (140)
      Module lists all update requests made by a client. If a client chooses to view a specific request, they can look into the request details module (141) and see any comments made by the law firm staff.
   Events Module (150)
      Module lists all set events for a client.
      Similar to the update request module, a client may choose to view a specific event which will provide further details of said event.
      Events are added by the client, the law firm, or by the system (if a deadline is generated from the workflow module).
      Client has the ability to confirm an event within the module.
   Documents Module (160)
      Module allows the client to view and download all documents available (i.e., documents uploaded by law firm staff from the law firm portal through the workflow module) as well as allow them to upload their own documents (which can be viewed by the client or the law firm).
   Rewards Module (180)
      Module lists every time the client has earned points and allows them to see their total points for a specific claim.
Law Firm Portal 200—(Dashboard)
   Home Module (210)
      Module allows a law firm to see a default dashboard of case statistics such as:
         Client Usage Metrics
         The metrics can be displayed as a graph (based on usage over a period of time) or in a table format
         Tracks app logins, pain journal entries, medical treatment check-ins, update requests, events scheduled/confirmed, etc.)
         Allows for advanced filters for demographics (filter by age, language, location, etc.)
         Task Progress
         The metrics can be displayed as a graph (based on usage over a period of time) or in a table format
         Tracks opened tasks, pending tasks, completed tasks, overdue tasks, and unassigned tasks.
         Allows for advanced filters (filter by client, claim, assignee, completion rate, overdue rate, time spent on, etc.)
         Most Recent Tasks
         Lists tasks recently worked on.
         Most Recent Activities
         Lists any claim activity done outside of tasks
         Update Requests
         Lists all update requests and their status
         Upcoming Events
         Lists upcoming events
         Claims/Users Statistics
         Shows general statuses of claims and users.
   Users Module (220)
      Module lists & gives access to all user groups as well as log history of all users.
         Groups
         Administrator, Staff, Clients, & Insurance Companies (3^{rd} Party), Medical Providers (3^{rd} Party), & Associated Counsel (3^{rd} Party)
   Matter Module (230)
      Modules lists & gives access to all claim-related sub-modules.
         Claims Listing Module—Add, delete, and view individual claims.
         Claim module 231.1 of the firm portal has access to claim module 100 of the client portal and its corresponding sub-modules of the law firm portal as follows:

| *Firm Portal | | ⁺Client Portal |
|---|---|---|
| Claim Module | Direction of Data Modification | Claim Module |
| Case Details | → | Case Details |
| 231.1a | Modified in FP*, changes updated to | 110 |
| | CP⁺ | |
| Raw Data | → | Case Details |
| 231.1b | Modified in FP, changes updated to CP | 110 |
| Clients | → | Case Details |
| 231.1c | Modified in FP, changes updated to CP | 110 |
| Medical | → | Case Details |
| 231.1d | Modified in FP, changes updated to CP | 110 |
| Contacts | → | Case Details |
| 231.1e | Modified in FP, changes updated to CP | 110 |
| 3 ^{rd} Party Info | → | Case Details |
| 231.1f | Modified in FP, changes updated to CP | 110 |
| Pain Journal | ← | Pain Journal |
| 231.1g | Modified in CP, changes updated to FP | 130 |
| Workflow | No direct transfer of data | |
| 231.1h | | |
| Tasks | → | Case Details |
| 231.1i | Modified in FP, changes updated to CP | 110 |
| Activities | No direct transfer of data | |
| 231.1j | | |
| Templates | No direct transfer of data | |
| 231.1k | | |
| Doc Repository | No direct transfer of data | |
| 231.1.81 | | |
| Client Docs | ← → | Documents |
| 231.1m | Data modified in either portal and | 160 |
| | changes updated to each other | |
| Shared Docs | → | Documents |
| 231.1n | Modified in FP, changes updated to CP | 160 |
| Update Request | ← → | Update Request |
| 231.1o | Data modified in either portal and | 140 |
| | changes updated to each other | |
| Treatment | ← → | Treatment |
| Calendar | Data modified in either portal and | Calendar |
| 231.1p | changes updated to each other | 120 |

Note:
the arrows correspond to the direction the data is
Tasks Listing Module (233) - Add, delete, and view individual tasks for a given claim.
Events Listing Module (234) - Add, delete, and view individual events for a specific user and claim.
Medical Listing Module (232) - Add, delete, and view medical status information for a specific user and claim

Master Settings Module (240)
   Module consisting of sub-modules that control different settings related to Matter modules as follows:
      Task Priority Settings (240.1)
      Pain Journal Settings (240.2)
      Modify response options for the pain journal entries
      Matter Data Settings (240.3)
      Modify data field settings
      Workflow Settings (240.4)
      Modify settings for the workflow, templates, and tasks
   Workflow/Template Settings System settings will be under this module.
Archived Module (250)
   Module which stores deleted case information which may be recovered.
System Settings Module (260)
   Module consisting of sub-modules that control website settings (i.e., appearance, server settings, displayed company info, chat availability.
Resources Module (280)
   Module which controls certain communications (i.e., text announcements, app announcements, standard system email, bulk emails).
Utilities Module (290)
   Module which allows a company to create backups, import data, & control system messages.
      Workflow/Template Settings System [within the Master Settings Module (240)]
Workflow/Template Admin Settings:
   The present invention comes with default workflow/templates that cannot be changed in the individual version, but allowed to in the business/enterprise version.
   A law firm has the ability to further customize templates with specific variables as placeholders to designate what information must be used for a given specific claim.
   A law firm has the ability to further customize workflows wherein they can add specific templates and/or customized tasks.
      Workflow/Template Module [within the Claim Module (231.1)]
Workflow/Template Module (231.1h):
   As shown in FIG. 11J, a user can choose the workflow category, the specific workflow, the client the workflow will be used for, and who will be the addressee.
      When choosing a workflow, data will be extrapolated from several modules as shown in FIG. 13F.
      Tasks with specific settings are generated along with documents prepopulated data that are associated with the tasks.
      Initially, all documents are exported to the document repository as drafts to be reviewed for thoroughness before the law firm exports the finalized version to the appropriate module.
         Thus, with the business/enterprise version of the client portal, a generated workflow is made for the specific claim and client where documents and tasks associated for said client will appear in the appropriate module in order to guide the law firm through the personal injury claim.

FIGS. 14A-14C illustrate diagrams and flowcharts for the client portal in an individual version 1200. All modules are identical as the modules in the business/enterprise version as they are the same claim management system and method. However, modules that individuals would have had as clients under the business/enterprise version are now editable by the individual (as there is no law firm managing the claim) and as such the numbering will remain the same. Modules numbered in the 600 series are derived from the modules in the law firm portal as they are functionally the same but now modified to be useable by a claimant directly. The following client portal is an outline for operation of the individual version.
Client Portal 010—As shown in FIG. 14A, a user will have access to the following modules:
   Claim Module (600)
      Lists all of a user's claims which they may select to view further.
      If no previous claim has been created, there will always be an option to fill out a guided form to create their first claim within the present invention.
   Chat Module (025)
      Allows a user to chat directly with an individual that was added on their contacts module 640 by establishing a temporary session.
   Notifications Module (050)
      Shows a user a list of notifications of certain actions within their cases. Notifications also list specific automated reminders (i.e., if a user has not check-in for treatment or made a pain journal entry for a specific period, the system will send them a reminder to do so).
   Settings Module (075)
      Allows a user to edit their profile or change password as well as access to specific support information.
Claim Module (600)—As shown in FIG. 14B, when a specific claim is chosen by a user, they will have access to the following modules for that claim:
   Case Details Module (110)
      Module allows a user to see their current case status as well as static data that was initially entered at the start of their claim
      They will have the option to further edit their case information as they work on their own case.
   Medical Treatment Calendar Module (120)
      A user will have the ability to track their treatment by the check-in option which will log the information. They will be able to use this information when reviewing medical records that will obtain once their treatment is complete.
      A user may also entered appointment information so the application may send them reminder notifications.
      A user may also enter further information about the medical providers they are seeing (i.e., treatment status, type of medical service, billing amounts)
   Pain Journal Module (130)
      Module lists all pain journals entry made by a user and allows them to add further entries as needed. A user may choose to view a specific entry for further information they added.
      They will be able to export all entries to serve as further documentation for their claim.
   Contacts Module (640)
      Allows a user to add contact information for all related parties (defendant, insurance companies, medical providers, health insurance, other claimants, witnesses, etc.)
   Events Module (150)
      Module lists all set events for a client which will include deadlines set by tasks generated from the workflow or events manually entered by the client
   Documents Module (160)
      Module allows the client to view and upload documents
   Workflow Module (670)
      A user can choose any number preset workflows for their claim which will generate the necessary tasks and documents associated with the task as shown in FIG, 14C
      Any documents generated will be automatically exported to the documents module (160) and give a user the option to share it by exporting it to the shared documents module.
   Tasks Module (680)
      A user will be able to see tasks generated by the workflow that will guide them through their personal injury claim to keep them on track.
   Shared Documents Module (690)
      A user will be able to share their documents and case information with other parties that they have permitted access to.
      Parties will be able to view and download documents based on restriction set by the user

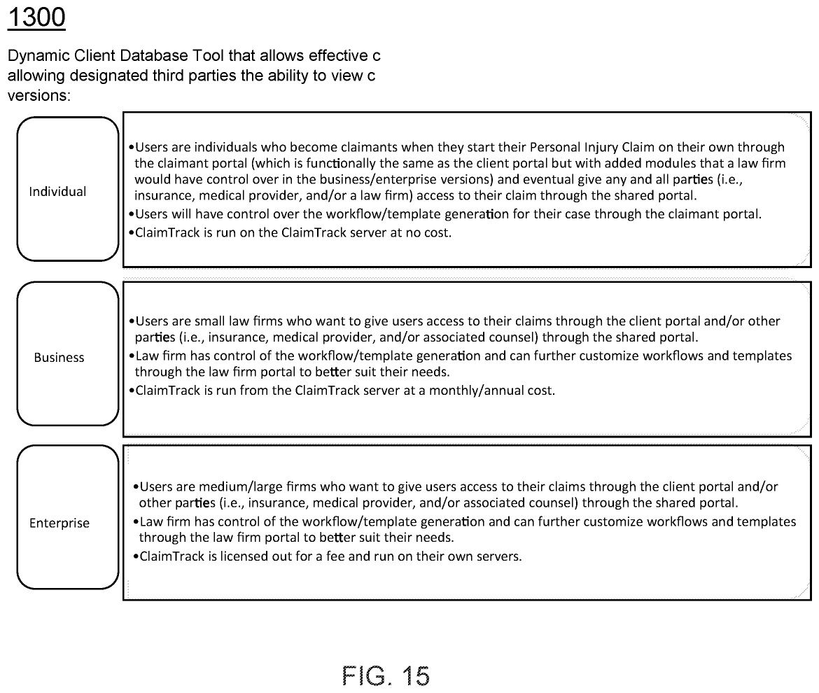

FIG. 15 illustrates a diagram of a Dynamic Client Database Tool that allows effective case processing and management while simultaneously allowing designated third parties the ability to view case progress and information. The users are classified in three tiers: Individual, Business, and Enterprise.

The Individual tier is for individuals who become claimants when they start their Personal Injury Claim on their own through the claimant portal (which is functionally the same as the client portal that is in the other two tiers but with added modules that a law firm would have had control over) and eventual give any and all parties (i.e., insurance, medical provider, and/or a law firm) access to their claim through the shared portal. As such, users will have control over the workflow/template generation for their case through the claimant portal. Further, the present invention is run on the server at no cost.

The Business tier is for small law firms who want to give users access to their claims through the client portal and/or other parties (i.e., insurance, medical provider, and/or associated counsel) through the shared portal. Further, law firm have control of the workflow/template generation and can further customize workflows and templates through the law firm portal to better suit their needs. Additionally, the present invention is run from the server at a monthly/annual cost.

The Enterprise tier is the same as the Business tier but geared toward medium/large firms who want full control and customization of the workflow/template generation by licensing the present invention running the application on their own servers.

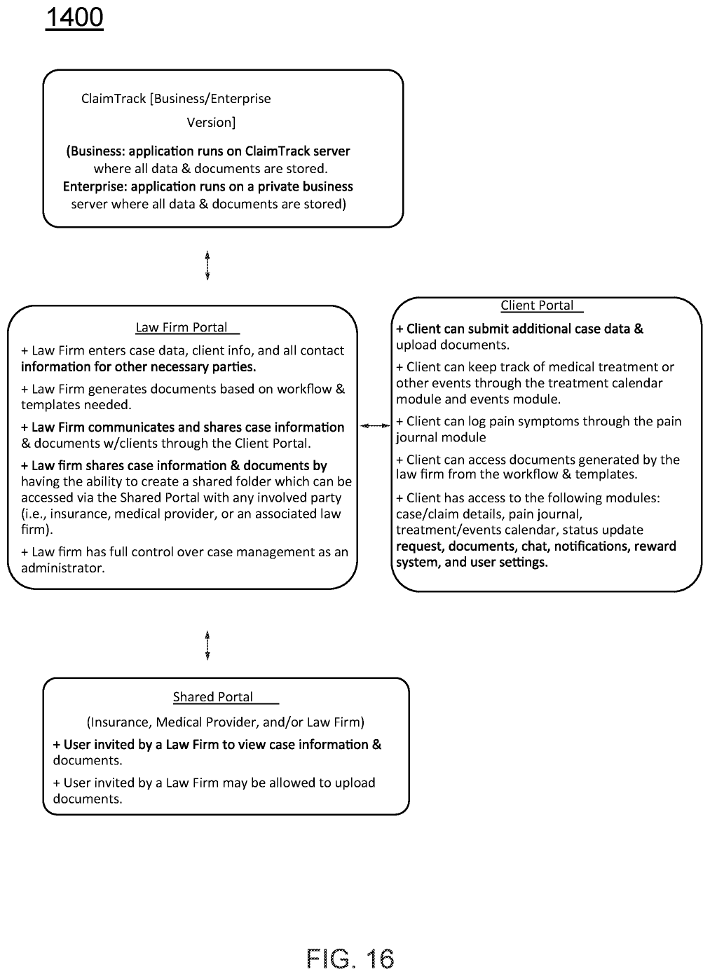

FIG. 16 illustrates a flowchart for the business and enterprise versions of the present invention. Initially, the law firm enters case data, client info, and all contact information for other necessary parties through the law firm portal. Next, the law firm generates tasks and documents based on workflow & templates needed. The law firm will further communicate and share case information & documents with their clients through the Client Portal. Further, the Client Portal can be used by clients to: (1) submit additional case information and upload further documents; (3) keep track of medical treatment through the treatment calendar; (4) keep track of other events through the events module; (5) create pain journal entries to keep track of their pain symptoms; (6) view/download documents generated by the law firm from the workflow; (7) view the progress of their case through the case details module; (8) request updates; (9) live chat with a case specialist during office hours; (10) receive various notifications to keep them informed and involved in their claim; and (11) receive points to be exchanged for rewards.

The law firm may also share certain case information and documents by creating a digital folder and sharing it by inviting any involved party (i.e., insurance, medical provider, or an associated law firm) to a shared portal where the parties may view, download, and/or uploading documents based on restrictions set by the law firm. As such, the law firm has full control over case management as an administrator.

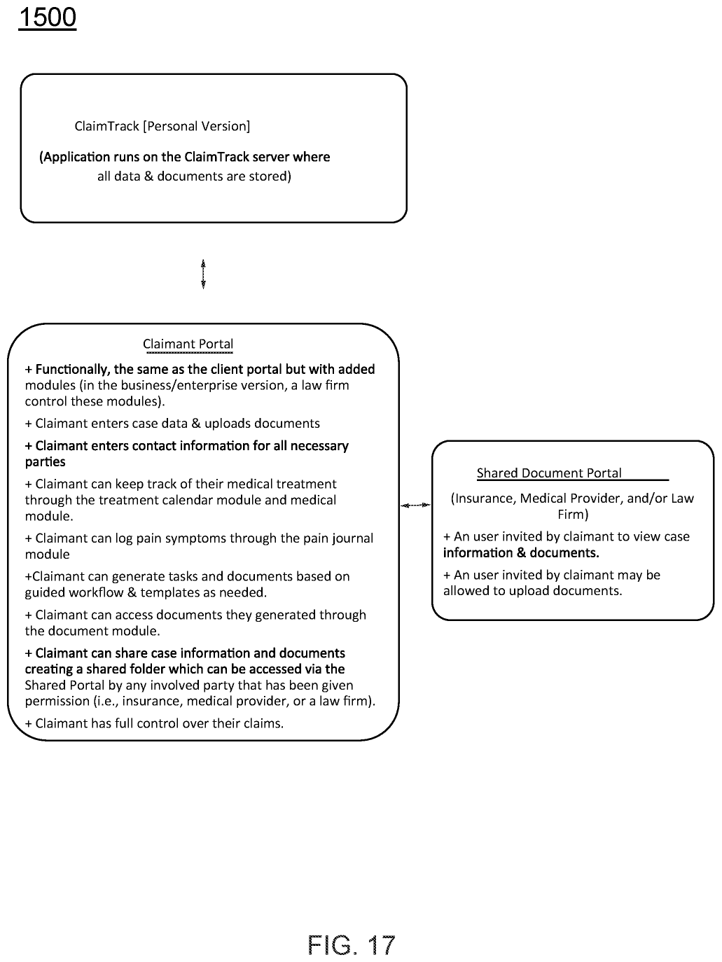

FIG. 17 illustrates a flowchart for the individual version of the present invention where the Client Portal functions similarly to the Client Portal in the Business/Enterprise tier except there are additional modules that were only useable by a law firm. Initially, the user as a claimant enters the claim information and upload their documents through the client portal directly. Next, the claimant will enter contact information and generate tasks and documents based on the workflow imported to keep them on track. Further, through the Client Portal, they will keep track of their medical treatment and information through the treatment calendar; (4) keep track of other events through the events module; (5) create pain journal entries to keep track of their pain symptoms; (7) view the progress of their case through the case details module as they complete tasks; (8) invite parties in their contacts module to a temporary live chat session; (10) receive various notifications to keep them informed and involved in their claim.

The claimant will also have the ability to share certain case information and documents by creating a digital folder and sharing it by inviting any involved party (i.e., insurance, medical provider, or a law firm) to a shared document portal where the parties may view, download, and/or uploading documents based on restrictions set by the claimant. As such, the claimant has full control over case management as an administrator.

In conclusion, a system 100 and method 1000 for processing legal claims in the personal injury industry, including medical and insurance related administrative procedures. More so, the claim management system and method provides an innovative portal for a user to digitally process a claim through guided workflows generating detailed tasks and documents tailored for that claim as well as tracking claim information and progress, facilitating open communications and document sharing between all involved parties to a claim. Through key modules within the claim management system and method, a robust framework is created to revolutionize personal injury industry standards through reductions in legal fees, decreases in claim duration, and ultimately, fostering an environment of transparency and trust between all parties at an unprecedented level.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

In summary, the present invention is a custom-designed software application that at its core is a multi-leveled system for improving the way personal injury claims, as well as the associated administrative tasks, are handled by attorneys, medical providers, insurance companies, and clients/claimants.

The first level of development within the present invention drastically improves the efficiency with which law firms handle and process all of their cases. This level includes the ability to customize document assembly, create corresponding workflows to track progress and understand the road ahead, as well as automate delivery of correspondence to relevant parties in an ongoing claim. Further, this level also includes the ability to set and assign tasks, communicate internally about status and progress, and make all current file information accessible by all team members, which accelerates efficiency through the collaborative platform built into this initial development.

The second level of development is the Client/Claim Portal, where clients can view all of their case information, including claim status, send communications to their legal representatives, access case documents, set and check claim-related calendar entries, track their medical treatment, create a pain history through their own pain journal and more, directly through their phone via the present invention mobile application. This creates a platform that allows clients to be active participants in their own claims—rather than just waiting passively for select information to be passed onto them by their legal representatives—and it fosters an environment of transparency between the law firm and client, since productive client oversight is now feasible and technology enabled.

The third level of development is how claim-related parties can access claim critical correspondence, documents, and other uploads, in a timely manner, keeping task organization as a collaborative effort, thereby preventing conflict in the later stages. By having a central repository of documents for all the moving parts, everyone has access to the same documentary resources, keeping claimants and liable parties on the same page, and creating transparency where previously there was only suspicion and assumption of malicious intent. This level of development also allows for efficiency in the claims handling process for all parties, as everyone will have the ability to share information, including client index information, claim status, provider information, insurance contacts, and more, with one another, reducing time delays from missed calls, unanswered emails, and simple procrastination.

The fourth level of development within the present invention is a combination of technical functions from the first and second levels, working in conjunction with the third level, which allows claimants to open, manage, and resolve their own insurance/injury claims. With the automated tools that the present invention has implemented, and best practices that have been set as claim parameters, claimants now have at their disposal resources and information that for too long has been kept as trade secrets by attorneys and insurance companies. Utilizing built-in custom workflows with tailored documents and templates, as well as attorney-created custom correspondence and claim forms, claimants can now make informed decisions on their own claims, without having to pay, or promise to pay portions of their recovery to, law firms, meaning that the ability for insurance companies to pay less while claimants recover more, is now a reality.

In the end, the present invention will change the way the personal injury practice area, and perhaps, the legal industry as a whole, handles client interaction, document drafting, and matter management. Clients and claimants, more than ever, need to be empowered, through information and technology, to make informed decisions about when they need legal services, how much legal services they really need, and how much to pay for those services. Movements underlying products such as the present invention will hopefully allow the free market to correct industry metrics through reductions in legal fees, decreases in claim duration, and more transparency in claim processing. Moreover, by putting power directly into the hands of the consumer claimants, we believe that the present invention will increase access to justice for all individuals (i) who were previously too intimidated by the legal process to initiate a claim; (ii) who are prone to hesitating too long because they don't know where to start or what questions to ask; or (iii) who may be too concerned about attorney fees and costs to actually pursue their legal rights.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. A method for processing legal claims in the personal injury industry, the method comprising:
accessing, by a claimant, a client portal through a software application, the claimant being involved in a personal injury incident;
recording, by the claimant, a personal injury case through a case detail module, recorded information documenting the personal injury incident;
scheduling a medical treatment calendar module to schedule medical treatment for the claimant;
documenting, by the claimant, physical and mental pain through a pain journal module;
communicating, between the claimant and a firm, through an update request module;
recording events related to the personal injury incident through an events module;
generating at least one document through a document module to generate workflow and documents between the claimant and another party such as an insurance company;
delivering, between the claimant, the insurance company, and the firm, the documents; and
awarding points to the claimant for utilizing the software application through a rewards module, the points being exchangeable for a prize;
wherein the case detail module, the medical treatment calendar module, the pain journal module, the update request module, the events module, the document module, and the rewards module are set in at least one processor.

2. The method of claim 1, wherein the claimant accesses the client portal 24/7.

3. The method of claim 1, wherein the document includes at least one of the following: a claimant medical record, a financial record, and an insurance claim.

4. The method of claim 1, further comprising:
viewing the documents by the claimant through a users module, and other involved parties through shared document portal, wherein the users module is also set in the at least one processor.

5. A claim management system for processing legal claims in the personal injury industry, the system comprising:
At least one processor configured to:
accessing, by a claimant, a client portal through a software application, the claimant being involved in a personal injury incident;
recording, by the claimant, a personal injury case, recorded information documenting the personal injury incident;
scheduling medical treatment for the claimant;
documenting, by the claimant, physical and mental pain;
communicating between the claimant and a firm;
recording events related to the personal injury incident;
generating at least one document to generate workflow and documents between the claimant and another party such as an insurance company;
delivering, between the claimant, the insurance company, and the firm, the documents; and
awarding points to the claimant for utilizing the software application, the points being exchangeable for a prize.

6. The system of claim 5, wherein the claimant accesses the client portal 24/7.

7. The system of claim 5, wherein the document includes at least one of the following: a claimant medical record, a financial record, and an insurance claim.

8. The system of claim 5, wherein the at least one processor further configured to viewing the documents by the claimant and other involved parties through shared document portal.
